(19) United States
(12) Patent Application Publication
(10) Pub. No.: US 2019/0059960 A1
(43) Pub. Date: Feb. 28, 2019

(54) TWO PART HAMMERTOE IMPLANT WITH EXPANDABLE TINES

(71) Applicant: Wright Medical Technology, Inc., Memphis, TN (US)

(72) Inventor: Jessica L. SHEMWELL, Millington, TN (US)

(21) Appl. No.: 16/071,521

(22) PCT Filed: Mar. 24, 2016
(86) PCT No.: PCT/US2016/023901

§ 371 Date: Jul. 20, 2018

(51) Int. Cl. A61B 17/72

(52) U.S. Cl. CPC A61B 17/7291; A61B 17/7266

(57) ABSTRACT

A two-part hammertoe system includes a double-sided hammertoe implant and a window capsule. The double-sided hammertoe implant includes a first end configured to couple the first end to a first bone and a second end comprising a plurality of expandable tines having a predetermined arrangement. The plurality of expandable tines are expandable from a first position having a first diameter to a second position having a second diameter. The second diameter is greater than the first diameter. The window capsule includes a closed distal end, a side wall extending proximally from the closed distal end, and a wedge extending from the distal end into an inner volume defined by the closed distal end and the side wall. The window capsule is sized and configured to couple to a second bone. The wedge is sized and configured to transition the plurality of tines from the first position to the second position.

### BACKGROUND

A hammertoe, or contracted toe, is a deformity of the proximal inter-phalangeal joint of the second, third, or fourth toe causing the toe to be permanently bent and giving the toe a semblance of a hammer. Initially, hammertoes are flexible and may be corrected with simple measures but, if left untreated, hammertoes may require surgical intervention for correction. Persons with hammertoe may also have corns or calluses on the top of the middle joint of the toe or on the tip of the toe and may feel pain in their toes or feet while having difficulty finding comfortable shoes.

One method of treatment may include correction by surgery if other non-invasive treatment options fail. Conventional surgery usually involves inserting screws, wires or other similar implants in toes to straighten them. Traditional surgical methods generally include the use of Kirschner wires (K-wires). K-wires require pings protruding through the end of respective toes due to their temporary nature. As a result, K-wires often lead to pin tract infections, loss of fixation, and other conditions. Additional disadvantages of K-wires include migration and breakage of the K-wires thus resulting in multiple surgeries. Due to the various disadvantages of using K-wires, however, compression screws are being employed as an implant alternative.

### SUMMARY

In various embodiments, a two-part hammertoe system including a double-sided hammertoe implant and a window capsule is disclosed. The double-sided hammertoe implant includes a first end configured to couple the first end to a first bone and a second end comprising a plurality of expandable tines having a predetermined arrangement. The plurality of expandable tines are expandable from a first position having a first diameter to a second position having a second diameter. The second diameter is greater than the first diameter. The window capsule includes a closed distal end, a side wall extending proximally from the closed distal end, and a wedge extending from the distal end into an inner volume defined by the closed distal end and the side wall. The window capsule is sized and configured to couple to a second bone. The wedge is sized and configured to transition the plurality of tines from the first position to the second position.

In various embodiments, a hammertoe implant is disclosed. The hammertoe implant includes a first end configured to couple the first end to a first bone and a second end including a plurality of expandable tines having a predetermined arrangement. The plurality of expandable tines are expandable from a first position having a first diameter to a second position having a second diameter. The second diameter is greater than the first diameter. The plurality of tines are transitioned from the first position to the second position when the second end is inserted into a window capsule coupled to a second bone. The window capsule comprises a closed distal end, a side wall extending proximally from the closed distal end, and a wedge extending from the distal end into an inner volume defined by the closed distal end and the side wall.

In various embodiments, a method for correcting hammertoe is disclosed. A first canal is formed in a first bone and a second canal is formed in a second bone. A first end of a hammertoe implant is inserted into the first canal in the first bone. The hammertoe implant has a second end including a plurality of expandable tines having a predetermined arrangement. The plurality of expandable tines are expandable from a first position having a first diameter to a second position having a second diameter. The second diameter is greater than the first diameter. A window capsule is inserted into the second canal in the second bone. The window capsule includes a closed distal end, a side wall extending proximally from the closed distal end, and a wedge extending from the distal end into an inner volume defined by the closed distal end and the side wall. The second end of the hammertoe implant is inserted into the window capsule to transition the plurality of tines from the first position to the second position by driving the plurality of tines into contact with the wedge.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates one embodiment of a two-part hammertoe implant including a double-sided hammertoe implant portion and an expanding cap portion.

FIG. 2 illustrates one embodiment of a double-sided hammertoe implant portion of a two-part hammertoe implant of FIG. 1 having a screw side and an expandable side.

FIG. 3A illustrates one embodiment of the expanding cap portion of the two-part hammertoe implant of FIG. 1.

FIG. 3B illustrates a cross-sectional view of the cap portion of FIG. 2A taken along line A-A.

FIG. 4A illustrates a second embodiment of the expanding cap portion of the two-part hammertoe implant of FIG. 1.

FIG. 4B illustrates an expanding end of a double-sided hammertoe implant configured to couple to the expanding cap portion of FIG. 3A.

FIG. 5 illustrates one embodiment of a two-part hammertoe implant coupled to a first bone and a second bone.

FIG. 6 illustrates one embodiment of a method of coupling a first bone and a second bone using the two-part hammertoe implant of FIG. 1.

### DETAILED DESCRIPTION

The description of the exemplary embodiments is intended to be read in connection with the accompanying drawings, which are to be considered part of the entire written description. In the description, relative terms such as “lower,” “upper,” “horizontal,” “vertical,” “above,” “below,” “up,” “down,” “top” and “bottom,” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

The present disclosure generally provides a two-part hammertoe implant for joining a first bone and a second bone, such as, for example, a proximal phalanx and a middle phalanx. The two-part hammertoe implant generally includes an implant portion and a cap portion. The implant portion includes a first end sized and configured for insertion into a first bone and a second end sized and configured for insertion into the cap portion. The cap portion is sized and configured for insertion into a second bone. After inserting the implant portion and the cap portion into respective first and second bones, the implant portion and the cap portion are coupled together to anchor the first bone to the second bone.

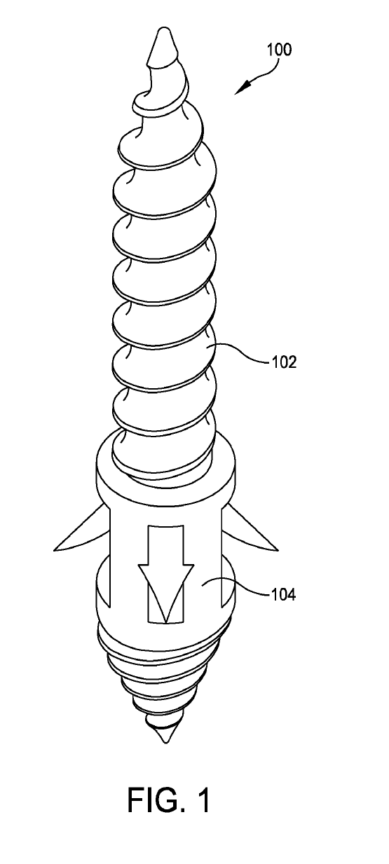

FIGS. 1-3B illustrate one embodiment of a two-part hammertoe implant 100 including a double-sided hammertoe implant 102 and window capsule 104. The double-sided hammertoe implant 102 includes a first end 106 and a second end 108 coupled by a shaft 110. The first end 106 is configured to anchor the double-sided hammertoe implant 102 to a first bone and the second end 108 is configured to anchor to a second bone. For example, in some embodiments, the first end 106 comprises at least one thread 112 configured to be received within a canal formed in a first bone. The at least one thread 112 can be configured to be inserted into a pre-drilled and/or pre-tapped canal and/or may comprise a self-drilling and/or self-tapping thread. The threaded section 112 can comprise a predetermined length to be fully implant into the first bone. In some embodiments, the first end 106 comprises one or more alternative and/or additional mechanism for coupling the hammertoe implant 102 to the first bone, such as, for example, a plurality of expandable arms. U.S. patent application Ser. No. 14/403,746, filed on Nov. 25, 2014, and entitled “Hammertoe Implant and Instrument” is incorporated herein by reference in its entirety.

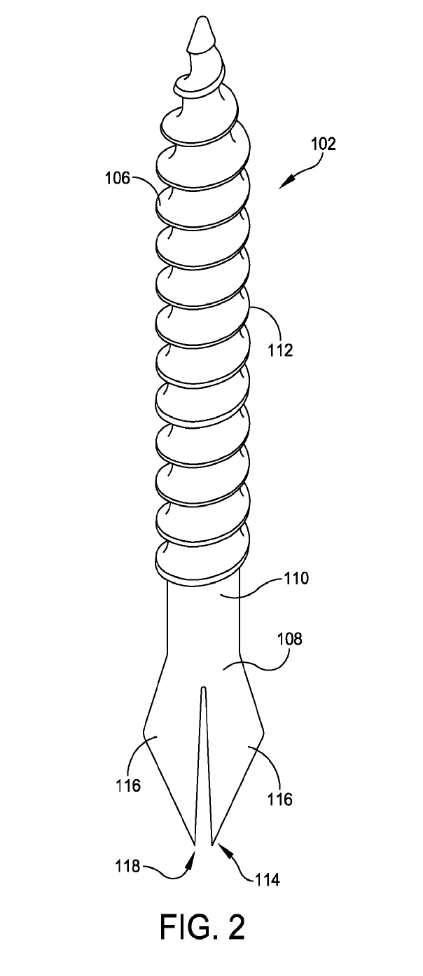

The second end 108 of the double-sided hammertoe implant 102 includes an expandable head 114 having a plurality of expandable tines or wings 116. The plurality of expandable tines 116 are coupled to the shaft 110 in a spaced-apart arrangement such that the plurality of expandable tines 116 define a central channel 118 therebetween. The plurality of tines 116 can comprise any suitable structure, such as, for example, a flat knife-blade structure, a geometric structure, and/or any other suitable structure. The plurality of expandable tines 116 are expandable from a first, collapsed position (shown in FIG. 2) to a second, expanded position (shown in FIG. 1) when the second end 108 is inserted into the window capsule 104. The tines 116 have a first diameter in the collapsed position and a second, larger diameter in the expanded position.

In some embodiments, at least a portion of the tines 116 is formed of a bendable and/or deformable material capable of deflecting the tines from the first position to the second position. For example, in some embodiments, at least a portion of the tines 116 comprises a metal material, plastic material, shape-memory material, and/or other suitable material capable of transitioning the tines from a first position to a second position when coupled to the window capsule 104. In some embodiments, the tines 116 can be transitioned from the second position to the first position for removal of the hammertoe implant 102 from the window capsule 104. In other embodiments, deformation of the tines 116 couples the hammertoe implant and the window capsule 104 in a permanent engagement.

The window capsule 104 comprises a body 120 having an open proximal end 122 and a closed distal end 124. The closed distal end 124 can comprise a point 127. The window capsule 104 is sized and configured for insertion into a canal formed in a second bone. In some embodiments, the distal end 124 of the body 120 defines one or more coupling mechanisms, such as, for example, a thread 125, for coupling the window capsule 104 within the canal of the second bone. In other embodiments, the window capsule 104 is within the canal by a friction fit.

The window capsule 104 has a sidewall 126 defining a plurality of openings 128 therein. In some embodiments, the plurality of openings 128 include a plurality of slots sized and configured to allow at least a portion of the tines 116 of the hammertoe implant 102 to pass therethrough. In other embodiments, the plurality of openings 128 can have any suitable shape and/or size configured to allow at least a portion of the tines 116 to pass therethrough. The side wall 126 can define a number of openings 128 greater, lesser, or equal to the number of tines 116. The window capsule 104 includes one or more features configured to transition the tines 116 from a first, collapsed position to a second, expanded position.

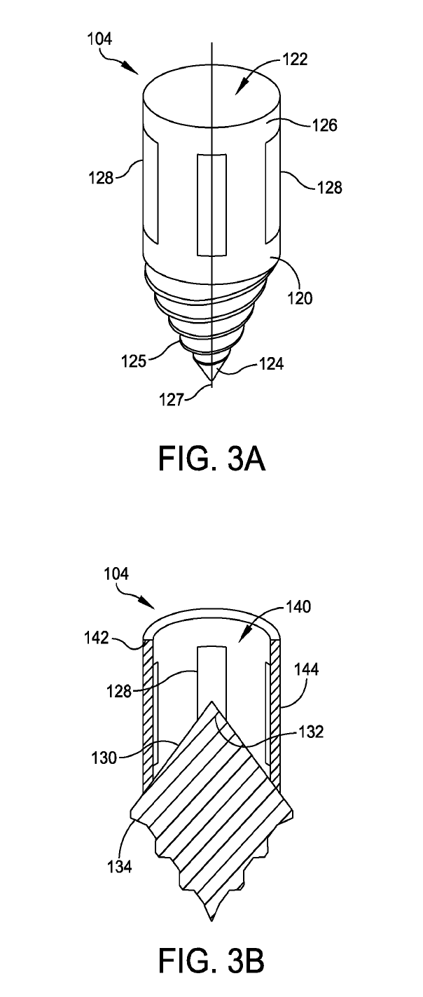

As best shown in FIG. 3B, in some embodiments, the window capsule 104 includes an internal wedge feature 130 sized and configured to transition the tines 116 from the first, collapsed position to the second, expanded position. The wedge feature 130 extends from a distal end 124 into an inner volume 140 defined by the side wall 126. In some embodiments, the wedge feature 130 includes a cone or other geometric shape that expands from proximal end to a distal end. For example, in the illustrated embodiment, the wedge feature 130 is a cone having a proximal apex 132 and a distal base 134. Although a cone-shaped wedge 130 is discussed herein, it will be appreciated that the wedge 130 can have any suitable shape, such as, for example, cone-shaped, pyramid-shaped, prism-shaped, tetrahedron-shaped, and/or any other suitable shape. The wedge 130 is positioned such that the tines 116 follow an upper surface 144 of the wedge and expand out of the openings 128 formed in the side wall 126.

The window capsule 104 has a diameter greater than the first diameter of the tines 116 but less than the second diameter of the tines 116. The second end 108 of the hammertoe implant 102 is sized and configured to be inserted into the open, proximal end 122 of the window capsule 104. When the second end 108 is inserted into the window capsule 104, the wedge feature 130 drives the tines 116 from a first, collapsed position (in which the tines 116 have a smaller diameter than the window capsule 104) to the second, expanded position (in which the tines 116 have a greater diameter than the window capsule 104). The tines 116 expand outward through the plurality of openings 128 to couple the second end 108 of the hammertoe implant 102 to the window capsule 104. In some embodiments, the tines 116 are configured to at least partially engage with an inner surface of a canal formed in the second bone. For example, in some embodiments, the tines 116 include a sharpened edge configured to cut or dig into at least a portion of the inner surface of the canal formed in the second bone.

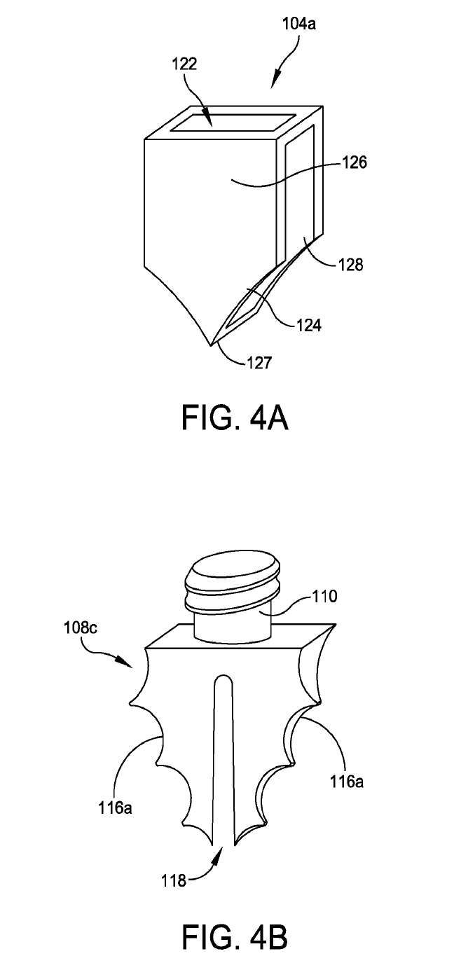

The shape of the window capsule 104 can be selected to match the shape and/or number of tines 116 formed on the hammertoe implant 102. For example, as shown in FIGS. 3A-3B, in some embodiments, the hammertoe implant 102 comprises at least four tines 116 defining a 3-dimensional diamond shape. The window capsule 104 has an open cylindrical shape sized and configured to receive the tines 116 therein and includes at least four openings 128 to allow passage of each of the tines 116 from the inner volume of the window capsule 104 to through the openings 128. As another example, as shown in FIGS. 4A-4B, in some embodiments, the hammertoe implant 102 comprises two tines 116a having a blade shaped configuration. The window capsule 104 has pyramid-shaped structure having two openings on opposing walls sized and configured to allow the tines 116a to pass from the inner volume through the openings 128. It will be appreciated that the second end 108 of the hammertoe implant 102 and the window capsule 104 can have any suitable complementary shape.

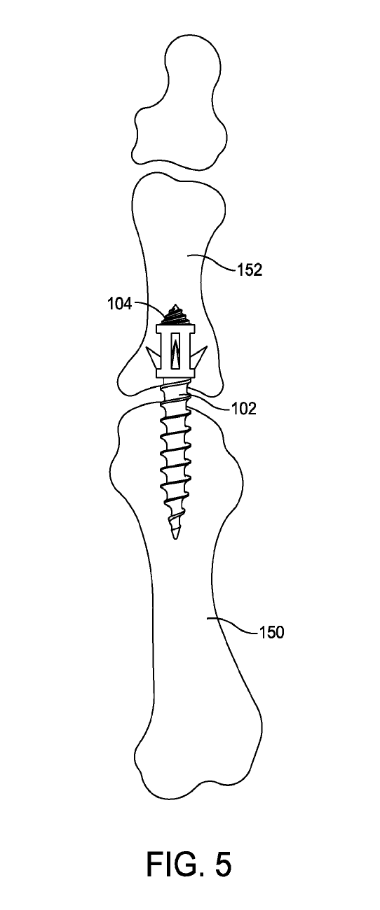

FIG. 5 illustrates one embodiment of a first bone 150 coupled to a second bone 152 by the two-part hammertoe implant 100. The first bone 150 and the second bone 152 can comprise inter-phalangeal bones of a toe. The first end 106 of the hammertoe implant 102 is inserted into a first bone 150. The window capsule 104 is coupled to the second bone 152. The first end 106 of the hammertoe implant and/or the window capsule 104 can be coupled to respective first and second bones by a threaded connection. The second end 108 of the hammertoe implant 102 is inserted into the window capsule 104 and the tines 116 of the second end 108 transition from a collapsed position to an expanded position. The tines 116 can expand through one or more openings 128 formed in the window capsule 104. The tines 116 maintain the hammertoe implant 102 in a fixed engagement with respect to the window capsule 104 such that a deformity of the first bone 150 and the second bone 152 is corrected. Although the two-part hammertoe implant 100 is illustrated with a first end 106 inserted into a first bone 150 and a second end 108 inserted into a second bone 152, it will be appreciated that the two-part hammertoe implant can be installed with the first part 106 inserted into the second bone 152 and the second part 108 inserted into the first bone 150.

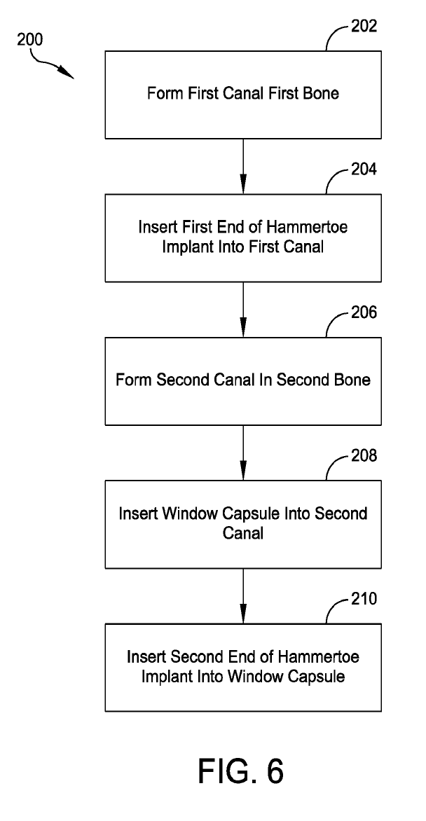

With reference to FIGS. 1-3B and 5-6, a method 200 for coupling a first bone 150 to a second bone 152 is disclosed. In a first step 202, a first canal is formed in the first bone. The first canal is sized and configured to receive a first end 106 of the hammertoe implant 102 therein. In a second step 204, the first end 106 of the hammertoe implant 102 is inserted into the first canal. For example, in some embodiments, the first end 106 includes at least one thread 112 disposed over a portion thereof. The at least one thread 112 is rotatably coupled to the first canal formed in the first bone 150. In some embodiments, the first canal can be pre-drilled and/or pre-tapped. In some embodiments, the at least one thread 112 includes a self-tapping and/or self-drilling thread. The thread 112 maintains the hammertoe implant 102 in a fixed engagement with respect to the first bone.

In a third step 206, a second canal is formed in the second bone 152. The second canal is sized and configured to receive a window capsule 104 therein. The window capsule 104 is inserted into the second canal in a fourth step 208. The window capsule 104 can include a threaded and/or pointed distal end 124 to facilitate insertion of the window capsule 104 into the second canal. For example, in some embodiments, the distal end 124 includes at least one thread 125 formed thereon. The window capsule 104 is rotatably inserted into the second canal to couple the at least one thread 125 to the inner wall of the canal. In some embodiments, the second canal is pre-drilled and/or pre-tapped. In some embodiments, the at least one thread 125 is a self-tapping and/or self-drilling thread. In other embodiments, the window capsule 104 is retained within the second canal by one or more additional and/or alternative coupling mechanisms, such as, for example, a friction fit.

In a fifth step 210, the second end 108 of the hammertoe implant 102 is inserted into the second canal. The second end 108 is pushed into the second canal to bring a plurality of tines 116 into contact with a wedge feature 130 formed within the window capsule 104. The wedge feature 130 drives the tines 116 from a first, collapsed position in which the tines 116 have a first diameter less than an inner diameter of the window capsule 104 to a second, expanded position in which the tines 116 have a second diameter greater than the outer diameter of the window capsule 104. The tines 116 expand through a plurality of openings 128 formed in a sidewall 126 of the window capsule 104. In some embodiments, the tines 116 include a cutting edge configured to cut and/or dig into the wall of the second canal. When the second end 108 is inserted into the window capsule 104, the first bone 150 and the second bone 152 are brought into a fixed engagement position to correct one or more defects, such as, for example, a hammertoe defect.

Although the subject matter has been described in terms of exemplary embodiments, it is not limited thereto. Rather, the appended claims should be construed broadly, to include other variants and embodiments, which may be made by those skilled in the art.

What is claimed is:

1. A system, comprising:
a double-sided implant comprising:
a first end configured to be coupled to a first bone;
a second end comprising a plurality of expandable tines having a predetermined arrangement, wherein the plurality of expandable tines are expandable from a first position having a first diameter to a second position having a second diameter, and wherein the second diameter is greater than the first diameter; and
a window capsule comprising a closed distal end, a side wall extending proximally from the closed distal end, and a wedge extending from the distal end into an inner volume defined by the closed distal end and the side wall, wherein the window capsule is configured to be coupled to a second bone, and wherein the wedge is sized and configured to transition the plurality of tines from the first position to the second position.

2. The system of claim 1, wherein the side wall of the window capsule defines a plurality of openings configured to receive the plurality of tines therethrough when the tines transition from the first position to the second position.

3. The system of claim 1, wherein the window capsule comprises at least one thread formed on an outer surface of the closed distal end.

4. The system of claim 1, wherein the plurality of tines comprise a three-dimensional diamond shape having an open central channel therebetween.

5. The system of claim 4, wherein the wedge comprises a cone-shaped wedge.

6. The system of claim 1, wherein each of the plurality of tines comprises a blade-shape having a serrated outer edge.

7. The system of claim 6, wherein the wedge comprises a pyramid-shaped wedge.

8. The system of claim 1, wherein the first end comprises at least one thread configured to couple the first end to the first bone.

9. The system of claim 1, wherein the hammertoe implant comprises a shaft extending longitudinally between the first end and the second end.

10. A hammertoe implant, comprising:
a first end configured to couple the first end to a first bone; and
a second end including a plurality of expandable tines having a predetermined arrangement, wherein the plurality of expandable tines are expandable from a first position having a first diameter to a second position having a second diameter, wherein the second diameter is greater than the first diameter, and wherein the plurality of tines are transitioned from the first position to the second position when the second end is inserted into a window capsule coupled to a second bone, the window capsule comprising a closed distal end, a side wall extending proximally from the closed distal end, and a wedge extending from the distal end into an inner volume defined by the closed distal end and the side wall.

11. The hammertoe implant of claim 10, wherein the plurality of tines are configured to expand through one or more openings formed in the side wall of the window capsule.

12. The hammertoe implant of claim 10, wherein the plurality of tines comprise a three-dimensional diamond shape having an open central channel therebetween.

13. The hammertoe implant of claim 10, wherein each of the plurality of tines comprises a blade-shape having a serrated outer edge.

14. The system of claim 10, wherein the first end comprises at least one thread configured to couple the first end to the first bone.

15. The system of claim 10, wherein the hammertoe implant comprises a shaft extending longitudinally between the first end and the second end.

16. A method for correcting hammertoe, comprising the steps of:
forming a first canal in a first bone;
forming a second canal in a second bone;
inserting a first end of a hammertoe implant into the first canal in the first bone, the hammertoe implant comprising a second end including a plurality of expandable tines having a predetermined arrangement, wherein the plurality of expandable tines are expandable from a first position having a first diameter to a second position having a second diameter, and wherein the second diameter is greater than the first diameter;
inserting a window capsule into the second canal in the second bone, the window capsule comprising a closed distal end, a side wall extending proximally from the closed distal end, and a wedge extending from the distal end into an inner volume defined by the closed distal end and the side wall; and
inserting the second end of the hammertoe implant into the window capsule to transition the plurality of tines from the first position to the second position by driving the plurality of tines into contact with the wedge.

17. The method of claim 16, comprising expanding the plurality of tines through a plurality of openings formed in a side wall of the window capsule.

18. The method of claim 17, transitioning the plurality of tines from the first position to the second position comprises inserting an apex of wedge into a central channel defined by the plurality of tines and driving the plurality of tines into contact with a tapered edge of the wedge to expand the plurality of tines to the second position.

19. The method of claim 16, wherein inserting the first end of the hammertoe implant into the first canal comprises threading at least one thread formed on the first end of the hammertoe implant into the first canal.

20. The method of claim 16, wherein inserting the window capsule into the second canal comprises threading at least one thread formed on a distal end of the window capsule into the second canal.
